(19) United States
(12) Patent Application Publication
(10) Pub. No.: US 2013/0239957 A1
(43) Pub. Date: Sep. 19, 2013

(54) ACTUATOR DEVICES FOR INHALERS

(71) Applicant: RELEVO LIMITED, Buckinghamshire (GB)

(72) Inventor: Andrew Pinfold, Belfast (GB)

(73) Assignee: RELEVO LIMITED, Buckinghamshire (GB)

(21) Appl. No.: 13/794,371

(22) Filed: Mar. 11, 2013

(30) Foreign Application Priority Data
Mar. 15, 2012 (EP) 12159749.6

(51) Int. Cl. A61M 15/00; A61M 11/04

(52) U.S. Cl. CPC A61M 15/0091; A61M 11/04
USPC 128/200.23

(57) ABSTRACT

An actuator for attachment to an inhaler of the type including a pressurized canister of medicine, the actuator comprising a sensor for detecting the presence of a patient to whom medicine is to be delivered from the pressurized canister; and the actuator comprising an electrically driven mechanical linear motion to trigger a lever which is moveable between an at rest position and an in use position in which the lever exerts a force on the canister so as to operate the inhaler and thereby release medicine from the pressurized canister to provide ease of use of the inhaler for patients with reduced or limited dexterity and grip/co-ordination. The actuator device can provide data logging using non-volatile internal memory; the data being capable of being downloaded via physical interface or Bluetooth.

### RELATED APPLICATIONS

This application claims priority and is entitled to the filing date of European Application Serial No. EP 12159749.6, filed on Mar. 15, 2012 and entitled “IMPROVEMENTS IN AND RELATING TO ACTUATOR DEVICES FOR INHALERS.” The contents of the aforementioned application are incorporated by reference herein.

### INCORPORATION BY REFERENCE

Applicant hereby incorporates herein by reference any and all patents and published patent applications cited or referred to in this application.

### BACKGROUND OF THE INVENTION

1. Field of the Invention

Aspects of this invention relate generally to inhalers, and more particularly to improved actuator devices for pressurised metered dose inhalers (PMDI's).

2. Description of Related Art

The following art defines the present state of this field:

U.S. Pat. No. 3,456,644 to Thiel et al. is generally directed to an aerosol dispensing device for discharging a metered amount of medicament-containing aerosol into the mouth of a patient during inhalation. The device is manually actuated to a charging condition and is latched in this position. A breath actuated trigger releases the latch and discharges a dose into the patient's mouth.

International Patent Application Publication No. WO 1992/007600 to Baum et al. is directed to an inhalation device for use with a pressurized aerosol canister containing a self-propelling medicament composition equipped with a dispensing valve having a stem movable relative to the canister between a closed position and a dispensing position, the device comprising a housing for supporting said canister and maintaining the valve stem in a fixed position relative to said housing in communication with a patient port, the device additionally comprising electromechanical means for moving said canister thereby actuating the dispensing valve for administration of medicament.

U.S. Pat. No. 5,133,343 to Johnson, IV et al. is generally directed to an apparatus having an outlet port insertable within a user's mouth, therein housed an automatically actuated commercially available and replaceable inhaler for discharging a medicated vapor through the outlet port into the user's mouth upon inhalation. The apparatus includes a housing having ribs for slidably supporting the inhaler, which inhaler is a cylindrical aerosol can containing medication under pressure. The housing is detachably attached to the apparatus to permit replacement of the inhaler and for cleaning purposes. A conduit extends from the housing for insertion into a user's mouth. On inhalation by a user, the apparatus will be actuated to slidably reposition the inhaler and effect discharge of a spray of medication through the conduit into the user's oral cavity and lungs.

U.S. Pat. No. 5,284,133 to Burns et al. is generally directed to an inhalation device with a mechanism to assure patient compliance with a drug dosage regimen. The control mechanism includes a controller, a timer, an actuator and a signaling device. The controller is programmed or preset with a time and dosage schedule for the drug to be delivered. For example, the controller may be programmed to allow for two puffs from an MDI every eight hours. The actuator operates in conjunction with the timer and prevents the inhalation device from being actuated after the programmed dosage has been administered at the prescribed interval. The actuator could be an electronically controlled valve or pawl arrangement or other suitable mechanism. The signaling device provides an audible, visual or tactile sensation during the time period prescribed for administration of the drug so that the patient is reminded to inhale his or her medicine at the prescribed time intervals. The history of actuation, non-actuation, and improper attempts at actuation can all be recorded and analyzed off-site at a later by a physician, pharmacist, or other authorized health care professional.

G.B. Patent No. 2389316 to Ross is generally directed to a dispensing system for biomedical use comprising a dispensing device operated under the control of a detachable control device. The control device may be removed and placed in a docking station connected to a control system with a user interface enabling a practitioner to control the dosing regime implemented using the device. Information concerning dosing is recorded in a database of the control device for downloading via the docking station of the control system. The dispensing device has an applicator such as a sublingual applicator, carrying sensors which are reactive to body fluids such as saliva in the user. The sensors may provide electrical signals to the control unit for use in regulating the dose or may alternatively be color indicators for signaling to the user or practitioner information relevant to the dosing regime. The dispensing device and control unit are each handheld and portable and may be reusable or disposable items.

U.S. Pat. No. 6,729,324 to Casper et al. is generally directed to a device for use with metered dose inhalers which includes a housing configured with a void to receive a metered dose inhaler, an actuator assembly which is configured to selectively apply force to the metered dose inhaler to cause the metered dose inhaler to release medicament, and a cocking mechanism for placing the actuator assembly in an armed configuration. The device is configured to actuate the metered dose inhaler as the user inhales, thereby ensuring improved medicament delivery. Additionally, the device is configured to prevent the metered dose inhaler from remaining in a vented position.

U.S. Patent Application Publication No. 2004/0231667 to Horton et al. is generally directed to a medicament dispenser comprising a medicament container having a dispensing mechanism; a container seat for receipt of the container; an anchor station; and drive means capable of moving the container seat relative to the anchor station to actuate the dispensing mechanism. The drive means is actuable in response to the application of non-mechanical energy thereto, and gear means are provided to the drive means to gear up the torque provided thereby.

G.B. Patent No. 2406283 to Jongejan et al. is generally directed to a compliance monitor attachable to or forming part of a drug delivery device, such as an inhaler. The monitor comprises a switch which is actuated by a user when delivering a dose of medicament. The monitor further comprises a sensor for sensing whether the device is properly positioned in contact with or relative to the user's body for administration of the medicament. For example where the device is an inhaler and the sensor a temperature sensor, temperature variations caused by insertion of the inhaler mouthpiece into the user's mouth indicate whether the dose has been delivered into the patient's mouth. A memory in the compliance monitor stores a compliance record indicating whether or not the device was properly positioned each time a dose was delivered. Also disclosed is a method of using a compliance monitor.

U.S. Patent Application Publication No. 2006/0137681 to Von Hollen et al. is generally directed to a metered dose inhaler which includes a canister fitted in an actuator body. A metered dose of medication is delivered by compressing the canister in the actuator body. The metered dose inhaler includes an actuator that either fully (automatic) or partially (user-assisted) actuates the metered dose inhaler in order to deliver medication to the user.

U.S. Patent Application Publication No. 2007/0240711 to Hamano is generally directed to a liquid ejection device that includes an inhaling port portion, an ejection head for ejecting a liquid to be inhaled by a user through the inhaling port portion, a first sensor for detecting an action of the user relating to inhalation, a second sensor for detecting another action of the user relating to inhalation, the second sensor being different from the first sensor, and a determining portion for determining an operation of ejecting the liquid of the ejection head in response to both a first signal output from the first sensor and a second signal output from the second sensor, wherein the determining portion enables ejection of the liquid from the ejection head when both the first signal and the second signal are signals indicating inhalation intention of the user.

International Patent Application Publication No. WO 2008/025087 to Flower is generally directed to a counter including a mounting portion for mounting the counter to an existing pressurized metered dose inhaler, an actuator movable relative to the mounting portion for actuating the inhaler when the counter is mounted to the inhaler, and an indicator which is driven in response to operation of the actuator so as to provide a visual indication related to an accumulated number of actuations of the inhaler.

U.S. Patent No. 2010/0228187 to Witte is generally directed to an applicator system with a pressurized medium and a valve for dispensing the medium, characterized in that the sprayhead has a sprayhead body, which includes an escape channel that is connected with the valve, and an outlet orifice in which the outlet channel empties, and a mouthpiece element is provided, which sits on the sprayhead body, whereas the outlet orifice and the mouthpiece element are arranged relative to each other, in order to bring the outlet orifice to the mouth of a user and to actuate the valve by the mouthpiece element.

U.S. Patent Application Publication No. 2011/0277764 to Terry et al. is generally directed to a personal vapor inhaling unit. An electronic flameless vapor inhaler unit that may simulate a cigarette has a cavity that receives a cartridge in the distal end of the inhaler unit. The cartridge brings a substance to be vaporized in contact with a wick. When the unit is activated, and the user provides suction, the substance to be vaporized is drawn out of the cartridge, through the wick, and is atomized by the wick into a cavity containing a heating element. The heating element vaporizes the atomized substance. The vapors then continue to be pulled by the user through a mouthpiece and mouthpiece cover where they may be inhaled.

By way of further background, and as illustrated by the above described prior art references, an inhaler comprising a housing holding a pressurized canister of atomized medicine for treatment of bronchial conditions such as asthma, is well known. Up until now, the use of such conventional inhalers requires a manual triggering of the inhaler for delivery of the drug through a mouthpiece. Conventional inhalers also rely on manually stored potential energy in a heavy spring to provide energy for the metered delivery of a measured amount of the drug. The ability to provide this function requires the patient to have enough physical strength and movement to prime the inhaler with potential energy and enough strength and movement to trigger the drug delivery. Patients of limited dexterity and limited motor function may have difficulty in achieving these functions. Also the prior art devices tend to require a high inspiration rate or a deep inspiration to ensure that the medication is released and delivered out of the inhaler. A huge problem associated with the prior art delivery devices is the tendency of the devices to result in delivery of the medication to be deposited at the back of the throat rather than delivered to the lungs of a patient, which is of course, the intended delivery location. For this reason, compliance with intended, prescribed medication delivery to the person's lungs tends to be a significant problem associated with the use of the prior art devices. Aspects of the present invention fulfill these needs and provide further related advantages as described in the following summary.

### SUMMARY OF THE INVENTION

Aspects of the present invention teach certain benefits in construction and use which give rise to the exemplary advantages described below.

The present invention solves the problems described above by providing an actuator device for engagement with an inhaler of the type including a pressurized canister of medicine, the actuator comprising detecting means for detecting the presence of a patient to whom medicine is to be delivered from the pressurized canister; and the actuator comprising an electrically driven mechanical linear motion mechanism adapted to operate a lever which is moveable between an “at rest” position and an “in use” position such that, in use, the lever exerts a force on the canister so as to operate the inhaler and release medicine from the pressurized canister to provide ease of use of the inhaler for patients with reduced or limited dexterity and grip/co-ordination.

Thus, the present invention provides a low voltage electronic means to trigger an electronic timed mechanism to activate a pressurised metered dose inhaler (pMDI) for the patient.

The actuator device of the present invention is a low-pressure triggered actuator device for releasing an atomised drug medicine in the mouth and consequently, in the air passage of a patient requiring delivery of the drug which is contained in the pressurised canister in the inhaler.

Ideally, the detecting means for detecting the presence of a patient comprises a sensor.

In a first embodiment, the sensor comprises a lip triggered sensor which is triggered when the sensor detects the presence of a person's lips in contact with a mouth piece of the inhaler.

In a second embodiment, the sensor comprises a breath activated sensor. The breath activated sensor may comprise a breath flow sensor or a vane sensor.

The actuator device is preferably provided in the form of a single unit for engagement with an inhaler.

The actuator advantageously has an electrically triggered lever to operate the inhaler. Preferably, the lever to operate the inhaler is driven by an electrically operated means comprising a servo, a solenoid or a gearbox.

The actuator device of the present invention is adapted for engagement with an inhaler comprising a housing and a pressurised canister. Advantageously, the means for adapting the actuator device for engagement with the inhaler comprise a mouth on the actuator device which is adapted to engage with the inhaler. The mouth of the actuator device ideally includes a seal so that the actuator device can sealingly engage with the inhaler.

Preferably, the actuator device mouth is shaped for clip-in and clip-out engagement with the inhaler so that the actuator device can be easily clipped into and out of engagement with the inhaler.

In one aspect, the actuator device can be clipped onto the mouthpiece of the inhaler. Preferably, in another aspect, the actuator device can be in the form of a housing which at least partially houses the inhaler when the inhaler is engaged with the actuator device and most preferably, when the inhaler is clipped-into and can be clipped-out of the housing defined by the actuator device.

Furthermore, the actuator device ideally comprises a mechanical counter display.

Preferably, the actuator device has a viewing window.

The actuator preferably includes a low power indicator.

The actuator device uses electrically driven mechanical linear motion to trigger the lever to operate the inhaler. Accordingly, the actuator device comprises means for operating the lever comprising a mechanical lever linkage operable to exert a force on the pressurised canister in the inhaler. Advantageously, the force exerted by the lever is a downward force so as to operate the inhaler, releasing medicine from the canister and delivering medicine to the user.

Preferably, the actuator comprises an internal non-volatile memory to facilitate usage recording.

The actuator advantageously comprises an internal timer circuit to control the activation of a lever to operate the inhaler.

The actuator ideally comprises a real time clock for itemized logging of activation.

The actuator ideally comprises an internal data log to record to record the number of times the inhaler has been used.

Optimally, the actuator includes means for downloading data logged in the non-volatile internal memory, said means comprising external interface or Bluetooth for extracting logged data and resetting.

The actuator advantageously has an external accessible battery compartment to enable changing of the battery.

Ideally, the present invention provides a low voltage actuator triggered by sensors so as to correctly operate an inhaler to facilitate patient compliance with appropriate inhaler use.

The actuator allows for battery exchange; and the actuator optimally has twelve months usage.

Preferably, the actuator device also comprises adjustment means for accommodating different sizes of inhaler in the actuator device housing. This adjustment means may comprise an insertable adjusting arm, or, alternatively, the adjustment means may comprise a plurality of insertable seals, each sized seal being insertable in the actuator device mouth such that an appropriate size of seal can be selected from the plurality of insertable seals and can be inserted to correspondingly match the size of the inhaler to be engaged with the actuator device.

In a first embodiment, the present invention provides an actuator for attachment to an inhaler, the actuator comprising a lip triggered sensor.

In the first embodiment, the actuator advantageously, is in the form of a mouthpiece which clips onto the inhaler mouthpiece such that the actuator mouthpiece is fitted onto the inhaler and the patient inserts the actuator mouthpiece into his/her mouth. Thus, when the lips come into contact with the lip sensor, the sensor sends a signal to the electrically powered unit to trigger the mechanical linkage and operate the lever so that the lever is moved into the in use position and the lever moves to exert a downward force on the pressurized canister so as to release medicine to the patient.

The actuator in the first embodiment may also comprise a sensor for detecting inhalation and/or exhalation to facilitate patient compliance of inhaler use with recommended delivery dose to the patient. Advantageously, the lip triggered sensor and the breath sensor may be contained within the mouthpiece. Ideally, the mouth piece is a wipe clean mouthpiece.

In the first embodiment of an actuator in accordance with the present invention, the triggering is to be mounted on a mouthpiece clip-on actuator device and comprises a capacitance based switch to reduce accidental discharge of the actuator device. On contact with the patient's lips, the sensor detects the presence of the patient's lips and the sensor then causes a triggering of an internal electronic timer circuit that controls a delay before actuating the actuation of an electronic triggered lever to drive the pMDI inhaler.

The timings of the delay of delivery will be determined by programmed variables. The timing variables are held in non-volatile memory and are available to be reprogrammed by an external contact interface. The device activation is also controlled by sensors to monitor breath direction in that the delay with continue until the patient is taking an inspiration.

This invention provides an actuator device in the form of an easy-grip wrapped plastic shell that serves as a holder for the internal electronics and a battery holder. In the first embodiment in which the actuator device comprises a lip sensor, the actuator device clips on, over the mouthpiece of a pMDI inhaler. The lip triggered sensor will commence the device's action of depressing the pMDI canister via linear mechanical electrically driven actuators. An internal log of the number of triggered deliveries will be held in non-volatile memory. An external mechanical counter is also provided to show the patient the number of deliveries per canister. This external counter is reset on insertion of the pMDI inhaler.

The patient reveals the mouthpiece by removing the mouthpiece protector, bringing the actuator device of the present invention, to the mouth and, on contact with the lips, the capacitance switch fires the timer circuit. The timer circuit provides a delay before drug delivery according to the patient's prescription, supplemented by an inspiration/expiration sensor to facilitate patient compliance. The sensor activates the actuator that levers the pressurized canister in the pMDI inhaler and the counter; the drug will be delivered into the patient's mouth. The switch will be disabled for a pre-determined period to prevent accidental discharge.

The configuration of the timer periods will be stored independently of the replaceable battery in non-volatile memory. The replaceable battery is used for the capacitance switch, timer circuit and actuators only. This will extend the life of the battery.

The timer circuit, capacitance switch connectors and mechanical actuators will be positioned within the main container. A battery level check is provided via an internal light emitting diode (LED) displayed through the lip switch insulators.

In a second and most preferred embodiment of the present invention, the actuator device is in the form of a housing into which an inhaler is inserted by clipping in the inhaler. Preferably, the actuator device housing includes an opening defining a mouth and the inhaler is inserted by clipping in the inhaler into the mouth of the actuator device housing. The actuator device mouth includes a seal around the mouth region to sealingly hold the inhaler. Thus, in the second embodiment, the actuator device is in the form of a housing into which an inhaler is inserted and at least partially housed such that the mouthpiece of the inhaler is accessible to the patient for insertion of the inhaler mouthpiece in the patient's mouth for drug delivery. Furthermore, in the second embodiment, which is the most preferred embodiment, the actuator device comprises a breath activated sensor.

In this, the most preferred embodiment of the present invention, the breath activated sensor is operable to activate the device at about one quarter of the way through an inhalation by a person who is using the device. Thus, the person using the device including the breath activated sensor places the mouth piece of the device to his/her mouth and inhales. At about one quarter of the way through that inhalation, the breath activated sensor detects the breath flowing past the sensor and activates the release of the medication so that in this way, as the person inhales, the medicine is released and is taken down into the lungs in a most effective manner i.e. the precisely prescribed amount of medication is delivered to the person's lungs because the device has been activated during the inhalation. The inhalation of air into the lungs carries the medicine down with the breath directly to the intended point of delivery i.e. the lungs and the medication is not ineffectively deposited at the back of a person's throat as tends to be the case with the prior art. A further advantage of the most preferred embodiment is that the device can be operated on a relatively low inspiration as well as a relatively high inspiration.

Advantageously, actuator device housing is adapted such that the inhaler is removeably inserted in the housing provided by the actuator device in a clip-in manner. Furthermore, the actuator device housing is adapted such that the inhaler is removable from the housing in an easily operated clip-out involving the user holding a lower portion of the inhaler or underneath the mouthpiece of the inhaler and only a relatively small force is needed to disengage the inhaler from the housing so that the inhaler is removed in a “clip-out” manner.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIGS. 1A and 1B are side views of an actuator device in a first embodiment of the present invention in which the actuator device includes a lip sensor; the actuator in this embodiment is in the form of a clip-on housing which clips onto the mouthpiece of a pMDI inhaler;

FIG. 2 is a front external view of the actuator device of FIG. 1 (i.e. first embodiment of the actuator device);

FIG. 3 is a rear external view of the actuator device in the first embodiment, showing the clips to house a pMDI inhaler. The internal view of mouthpiece is shown;

FIGS. 4A and 4B are schematic diagrams of the internal mechanical actuators of the actuator device in the first embodiment of the present invention;

FIG. 5 is a perspective view of the actuator device in a second embodiment of the present invention engaged with an inhaler;

FIG. 6 is a front view of the actuator device in the second embodiment, engaged with the inhaler;

FIG. 7 is a first side view from one side of the actuator device of FIG. 5;

FIG. 8 is a second side view from the other side of the actuator device of FIG. 5;

FIG. 9A is a plan view from above the actuator device of FIG. 5 engaged with an inhaler;

FIG. 9B is a plan view from underneath of the actuator and inhaler of FIG. 5;

FIG. 10A is a sectional view showing the internal mechanism for activation of the pressurized canister in the inhaler which is engaged with the actuator device in use;

FIG. 10B is an exploded sectional view of the internal mechanism of FIG. 10A;

FIG. 11 is a side view of the actuator device in the second embodiment, shown without an inhaler inserted in the actuator device;

FIG. 12 is a front view of the actuator device of FIG. 11;

FIG. 13A is a side view of the actuator device as shown in FIG. 11 but in partial section, showing an insertable adjuster means for adjusting the size within the housing so as to be capable of engagingly receiving a smaller sized inhaler;

FIG. 13B is a front view of the actuator device as shown in FIG. 13A;

FIG. 13C is a side view of the insertable adjuster means shown by itself, removed from the actuator device of FIG. 13A; and

FIG. 13D is a front view of the insertable adjuster means shown removed from the actuator device of FIG. 13B.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION OF THE INVENTION

The above described drawing figures illustrate aspects of the invention in at least one of its exemplary embodiments, which are further defined in detail in the following description.

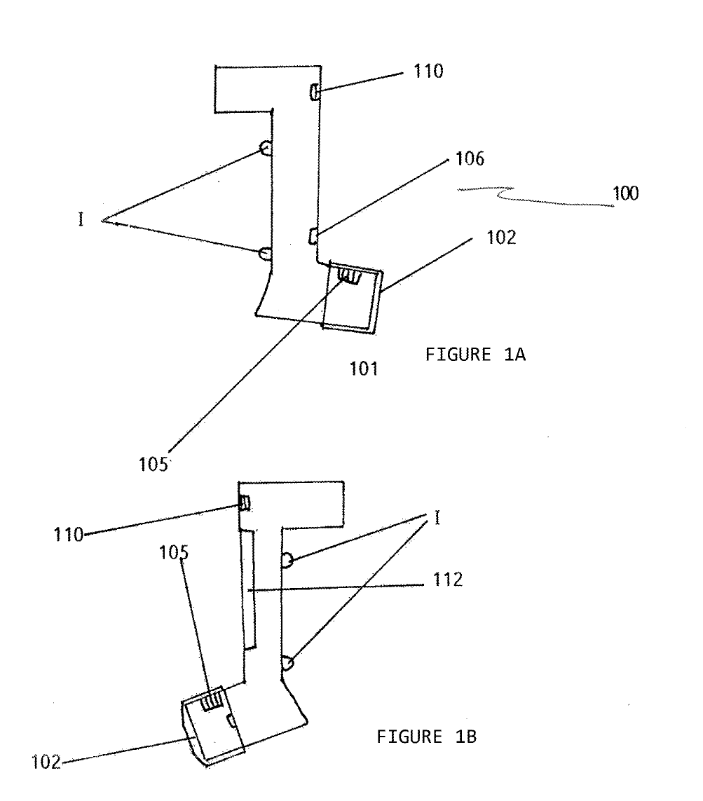

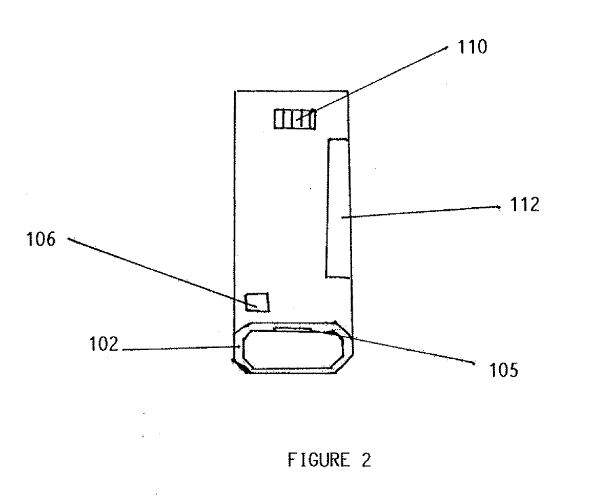

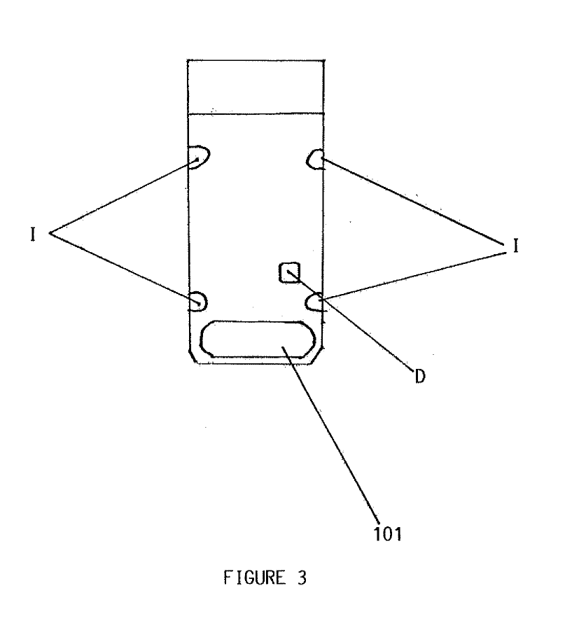

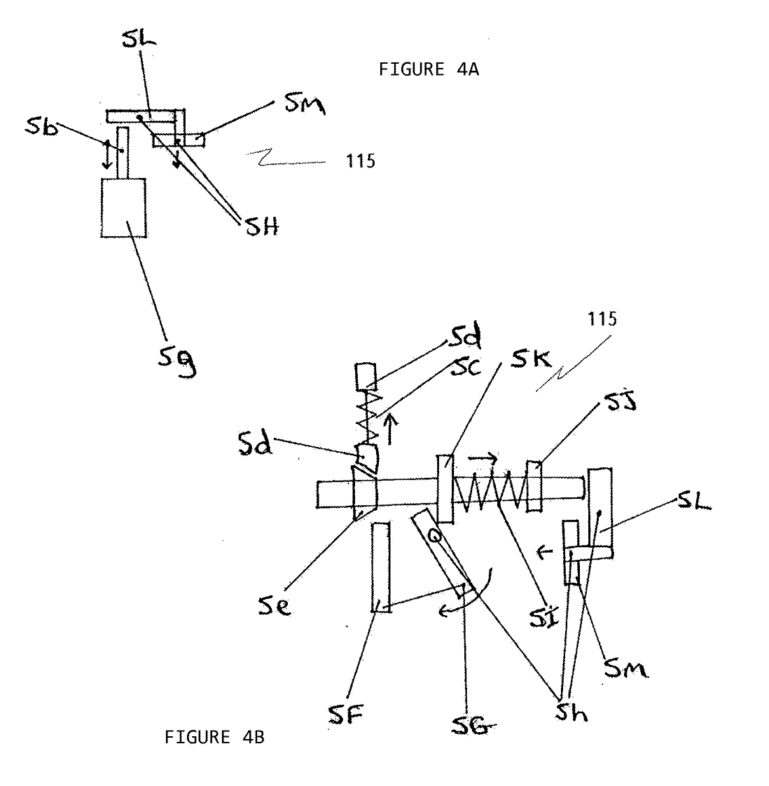

Referring initially to FIGS. 1 to 4 inclusive, of the drawings, the actuator device in the first embodiment will be described. The actuator device in the first embodiment is indicated generally by reference numeral 100.

An inhaler I, comprising a housing and a pressurised canister, can be engaged with the actuator device 100. The actuator device 100 includes a lip sensor 105 is provided on the mouthpiece 101, mouthpiece protector cover 102, counter viewing window 106, the external interface 110 and the battery access cover 112.

The lever mechanism is indicated generally by reference numeral 115. The manner of operation of the actuator in the first embodiment will now be described. The voltage supply at start delivery 5f and 5g causes a lever pivoted at 5h to compress the return spring 5i to engage the arm attached at 5k to slide through 5j until the arm attached 5e is pulled past lock 5d. The return spring 5c will engage the lock 5d behind 5e and hold the arm that engages 5l pivoting arm which pushes down 5m base plate. At the time cessation control point as determined by the timer is reached, a voltage is applied to 5a and 5b which contracts the actuator at 5c to release the fixed point 5e and the arm is returned by the return spring 5i. The lock 5d is returned by the spring 5c. Case fixed points are 4a, 5b, 5f, 5h, 5.

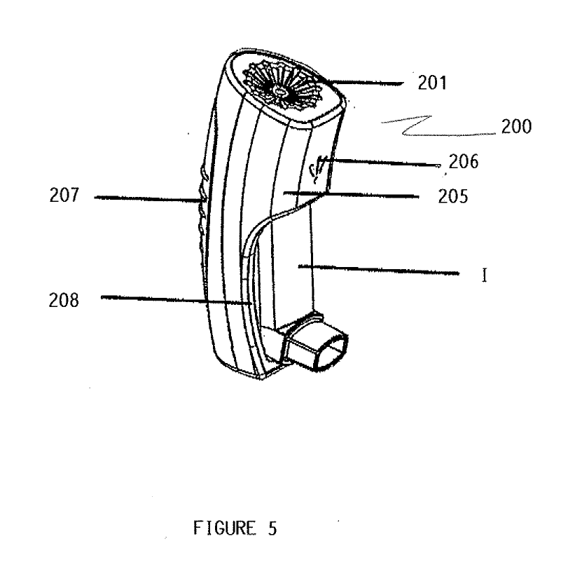

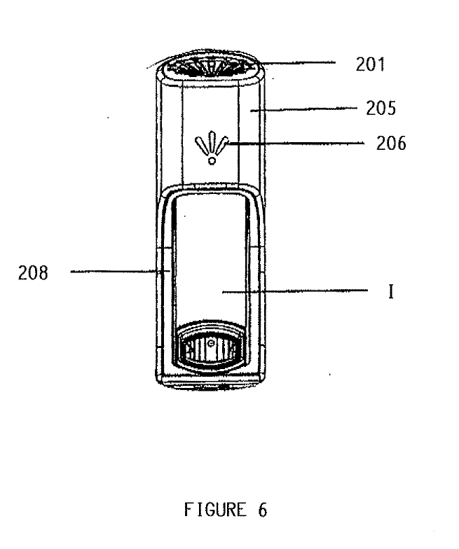

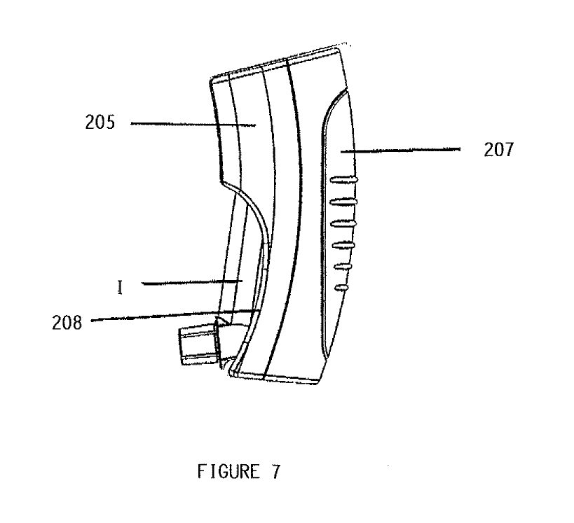

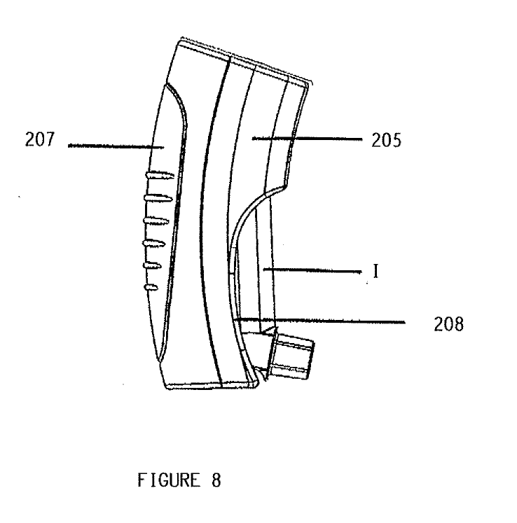

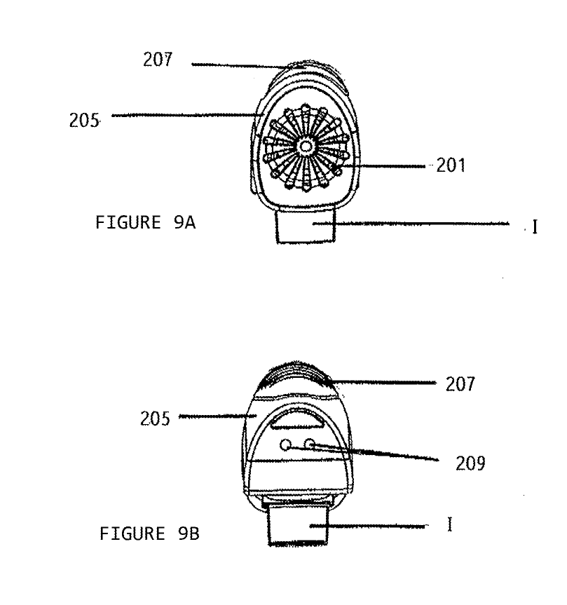

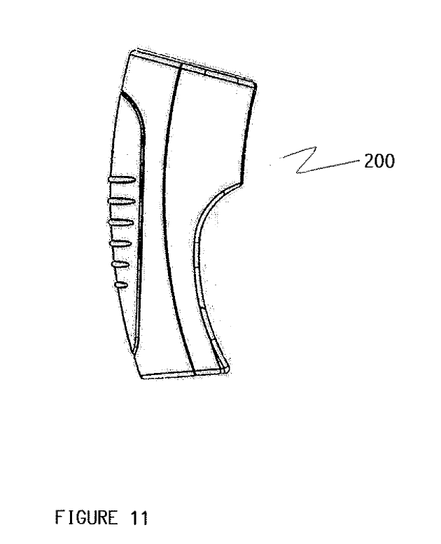

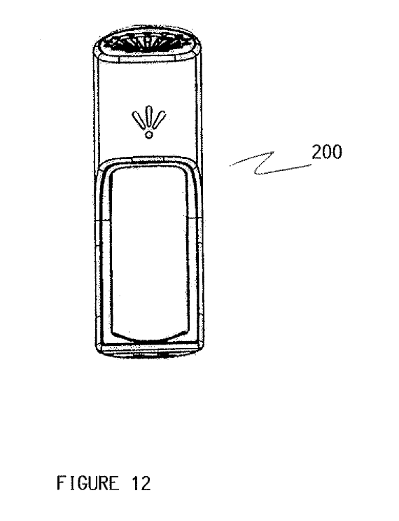

Referring now to FIGS. 5 to 13 inclusive, the actuator device in the second embodiment will now be described. The actuator housing in the second embodiment is indicated generally by reference numeral 200.

In FIGS. 5 to 10, the actuator device 200 is shown ready for use, with an inhaler I (comprising an inhaler housing and a pressurised canister) engaged in the actuator device 200.

Referring to FIGS. 5 to 13D, the actuator 200 includes the following features:

Air vents 201, plastic housing 205, LED light 206, pMDI inhaler I, seal 208, rear hand grip 207 and battery charging points 209.

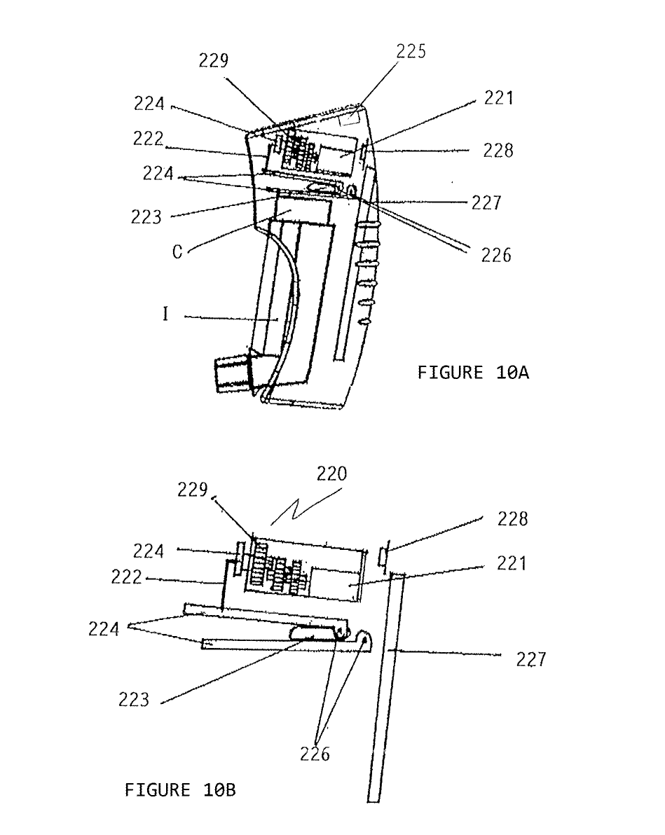

Referring now to FIG. 10A and FIG. 10B, the sectional view in FIG. 10A and exploded sectional view of lever linkage in FIG. 10B, the general reference numeral for the mechanism is 220; and the mechanism 220 includes an electrically driven linear motor 221 (e.g. servo, gearbox or solenoid), linkage 222, plastic cam 223; and lever arms 224.

A pMDI canister inhaler A including canister C, lever fixing pins 226, lithium ion battery 227, PCB board 228 and gears 229.

The lithium ion battery 227 lasts up to 1 year. The lithium ion battery 227 may be rechargeable. If it is a rechargeable battery, then the two recharging contact points 209 enable power to flow into the battery when placed in a re-charging unit (not shown in the drawings).

Furthermore, in the second embodiment, the actuator device is in the form of a housing into which an inhaler is inserted and at least partially housed such that the mouthpiece of the inhaler is accessible to the patient for insertion of the inhaler mouthpiece in the patient's mouth for drug delivery.

The actuator device housing is adapted such that the inhaler is removeably inserted in the housing provided by the actuator device in a clip-in manner. Furthermore, the actuator device housing is adapted such that the inhaler is removable from the housing in an easily operated clip-out involving the user holding a lower portion of the inhaler or underneath the mouthpiece of the inhaler and only a relatively small force is needed to disengage the inhaler from the housing so that the inhaler is removed in a “clip-out” manner.

FIGS. 10A and 10B are section views showing the internal electrical servo gearbox 221, linkage bar 222, lever arms 224, plastic cam 223, pMDI canister C, sensor 225, lever pivoted pins 226, servo or gearbox fixing points 226.

Referring to FIGS. 5 to 10, the operation of the device will now be described. In use, an inhaler I is clipped into the housing defined by the actuator device 200 and the mouthpiece of the inhaler will be exposed as shown in FIG. 10A. The user positions his/her mouth over the inhaler mouthpiece and inhales. Because of this inhalation, air is drawn in through the air vents 201 and this air flow activates the breath flow sensor 225.

The activation of breath flow sensor 225 or vane sensor, in turn, causes activation of an electrically driven gearbox servo or gearbox 221 which pushes the linkage bar 222 and thereby pushes the lever arms 224 so as to compress the pMDI inhaler canister C to release drug so as to deliver the drug dosage in the optimum way.

Thus the breath flow sensor 225 is operable to activate the electrically driven gearbox servo or gearbox 221 at about one quarter of the way through an inhalation by a person who is using the actuator device 200. Thus, the person using the actuator device 200 including the breath flow sensor 225 places the mouth piece of the actuator device 200 to his/her mouth and inhales. At about one quarter of the way through that inhalation, the breath flow sensor 225 detects the breath flowing past the sensor 225 and activates the electrically driven gearbox servo or gearbox 221 which pushes the linkage bar 222 and thereby pushes the lever arms 224 so as to compress the pMDI inhaler canister C to release drug so as to deliver the drug dosage in the optimum way.

In this way, as the person inhales, the medicine is released and is taken down into the lungs in a most effective manner i.e. the precisely prescribed amount of medication is delivered to the person's lungs because the device has been activated during the inhalation. The inhalation of air into the lungs carries the medicine down with the breath directly to the intended point of delivery i.e. the lungs.

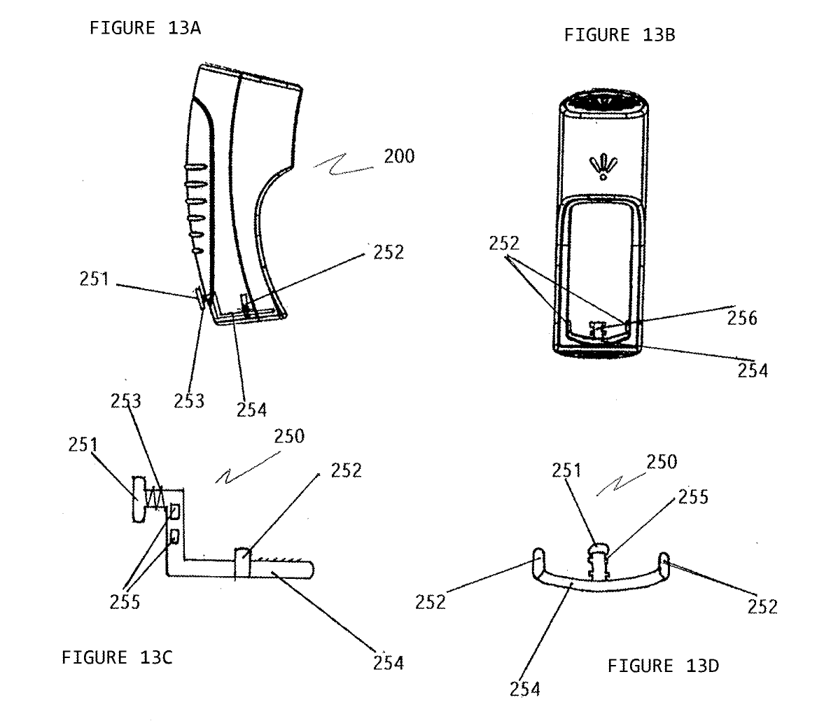

Referring now to FIGS. 13A to 13D inclusive, an insertable adjusting arm for adjusting the available height within the housing defined by the actuator device 200 so as to accommodate differently sized pMDI inhalers, is shown and is indicated generally by reference numeral 250.

The insertable adjusting arm 250 includes the following features:

Rear push in slide button to adjust height 251, side stabiliser sliding peg 252, button ejecting spring 253, base plate with grips to hold a pMDI inhaler 254, rear locating teeth 255; and rear locating locking points for height adjustment of a pMDI inhaler 256.

Features and advantages of the actuator device 220 include the following:

A portable, battery operated, breath activated pMDI inhaler clip-in attachment is provided by the present invention, for ease of use for patients with reduced or limited dexterity and co-ordination;
electrically driven breath activated clip-in attachment for a pMDI inhaler;
clip-in insertion of the inhaler in the actuator device in the second embodiment and clip-out withdrawal of the inhaler from the actuator device housing in the second embodiment of the actuator device of the present invention; and
clip-on attachment of the actuator device in the first embodiment whereby the actuator device in the first embodiment is engaged with the mouth piece of the inhaler; and
the actuator device in any embodiment of the present invention can engage with most pMDI thereby providing simpler and easier activation and use of any pMDI.

To summarize, regarding the exemplary embodiments of the present invention as shown and described herein, it will be appreciated that improved actuator devices for pressurised metered dose inhalers are disclosed. Because the principles of the invention may be practiced in a number of configurations beyond those shown and described, it is to be understood that the invention is not in any way limited by the exemplary embodiments, but is generally directed to improved actuator devices for pressurised metered dose inhalers and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention. Furthermore, the various features of each of the above-described embodiments may be combined in any logical manner and are intended to be included within the scope of the present invention.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. An actuator device for engagement with an inhaler of the type including a pressurized canister of medicine, the actuator comprising detecting means for detecting the presence of a patient to whom medicine is to be delivered from the pressurized canister; and the actuator comprising a mechanical linear motion to operate a lever which is moveable between an “at rest” position and an “in use” position in which, in use, the lever exerts a force on the canister so as to operate the inhaler and release medicine from the pressurized canister.

2. The actuator device of claim 1 wherein the means for detecting the presence of a patient comprises a sensor.

3. The actuator device of claim 2 wherein the sensor is a lip triggered sensor which is triggered when the sensor detects the presence of a person's lips in contact with a mouth piece of the inhaler; optionally wherein the lip triggered sensor is provided on the actuator device which is in the form of a housing which, in use, connects onto the mouth piece of the inhaler.

4. The actuator device of claim 2 wherein the sensor comprises a breath activated sensor.

5. The actuator device of claim 1 having a clip-in connection for engaging the actuator with the inhaler; optionally wherein the actuator device is in the form of a housing which at least partially houses the inhaler when the inhaler is engaged with the actuator device and particularly, when the inhaler is clipped-into the housing defined by the actuator device.

6. The actuator device of claim 1 wherein the lever to operate the inhaler is driven by an electrically operated means; optionally wherein the electrically operated means comprises a servo, a solenoid or a gearbox.

7. The actuator device of claim 1 having a mechanical counter display; optionally the actuator device having a viewing window.

8. The actuator device of claim 1 having a second sensor for detecting inhalation and/or exhalation to facilitate patient compliance of inhaler use with recommended drug delivery dosage to the patient.

9. The actuator device of claim 8 wherein the lip triggered sensor and the sensor for detecting inhalation and/or exhalation are provided on the actuator device which is in the form of a housing which, in use, connects onto the mouth piece of the inhaler; optionally wherein the mouth piece is a wipe clean mouthpiece.

10. The actuator device of claim 1 wherein the means for adapting the actuator device for engagement with the inhaler comprise a mouth on the actuator device which is adapted to engage with the inhaler; optionally wherein the mouth of the actuator device includes a seal so that the actuator device can sealingly engage with the inhaler.

11. The actuator device of claim 1, wherein the actuator device uses electrically driven mechanical linear motion to trigger the lever to operate the inhaler.

12. The actuator device of claim 1, wherein the actuator device comprises an internal non-volatile memory to facilitate usage recording; optionally wherein the actuator device comprises an internal timer circuit to control the activation of a lever to operate the inhaler; optionally wherein the actuator device comprises a real time clock for itemised logging of activation; optionally wherein the actuator device comprises an internal data log to record to record the number of times the inhaler has been used; optionally wherein the actuator device comprises means for downloading data logged in the non-volatile internal memory, said means comprising an external interface or Bluetooth for extracting logged data and resetting.

13. The actuator device of claim 1, wherein the actuator is a low voltage actuator triggered by a sensor so as to correctly operate an inhaler to facilitate patient compliance with appropriate inhaler use; optionally wherein the actuator includes a low power indicator.

14. The actuator device of claim 1 including means for enabling battery exchange; optionally wherein the actuator device comprises an external accessible battery compartment to enable changing of the battery; and optionally wherein the battery provides twelve months usage.

15. The actuator device of claim 1 wherein the actuator device also comprises adjustment means for accommodating different sizes of inhaler in the actuator device housing; optionally wherein said adjustment means comprises an insertable adjusting arm; or optionally wherein the adjustment means comprises having a plurality of insertable seals, insertable in the actuator device mouth such that an appropriate size of seal can be inserted to correspondingly match the size of the inhaler to be engaged with the actuator device.
